(19) United States
(12) Patent Application Publication
(10) Pub. No.: US 2007/0053969 A1
(43) Pub. Date: Mar. 8, 2007

(54) Device for treating and promoting healing of damaged body tissue

(76) Inventor: Michael F. Oyaski, Ebensburg, PA (US)

Correspondence Address: JAMES RAY & ASSOCIATES, 2640 Pitcairn Road, Monroeville, PA, 15146, US

(21) Appl. No.: 11/546,219

(22) Filed: Oct. 11, 2006

Related U.S. Application Data
(62) Division of application No. 10/790,318, filed on Jun. 1, 2004.

(51) Int. Cl. A61K 9/70

(52) U.S. Cl. 424/449

(57) ABSTRACT

A device for treating and promoting healing of damaged body tissue. The device comprises an encapsulating device disposed adjacent the damaged tissue for controlling the environmental conditions surrounding the tissue. A transfer device is disposed on the encapsulating device to allow at least one of a predetermined medium to communicate with the encapsulating device without removing the encapsulating device. The damaged tissue can be effectively treated and healed in controlled environmental conditions.

### FIELD OF THE INVENTION

The present invention relates, in general, to a device for treating and promoting healing of damaged body tissue, and more particularly, to a device for effectively treating and healing damaged body tissue in controlled environmental conditions.

### BACKGROUND OF THE INVENTION

Prior to the present invention, medical personnel routinely treat second and third degree burns, and skin grafts performed on burns by applying a topical antibiotic and sterile gauze bandage to the affected areas. New gauge bandages and antibiotic are applied daily. Body fluids flow from the damaged skin and dry on the bandage, making bandage removal a very painful procedure for the patient. Additionally, this procedure can interrupt and significantly slow the healing process. To prevent the affected area from drying out, the bandage must be continuously moistened. Burned tissue also feels very hot and is extremely sensitive to temperature changes. Because burns covering over 75% of the body typically result in death due to the loss of bodily fluids, the patient must be continuously rehydrated to replenish essential bodily fluids.

Medical personnel also treat severe wounds by suturing, applying antibiotics, and covering with a gauge bandage to protect the affected area during the healing process. Bodily fluids that seep from the wound adhere the gauze bandage to the wound, causing pain to the patient when the bandage is removed. In deep wound situations, standard emergency procedure is to apply a tourniquet to the affected area to restrict the loss of blood. However, a tourniquet can also damage healthy tissue by restricting the blood flow, therefore the tourniquet must be periodically loosened to prevent tissue damage and subsequent infection.

### SUMMARY OF THE INVENTION

In a first aspect, the present invention provides a device for treating and promoting healing of damaged body tissue. The device comprises an encapsulating means disposed on the tissue for controlling the environmental conditions surrounding the tissue. A transfer means is disposed on the encapsulating means to allow at least one of a predetermined medium to communicate with the encapsulating means without removing the encapsulating means. The tissue can be effectively treated and healed in controlled environmental conditions.

In a further aspect, the present invention provides a device for treating and promoting the healing of damaged body tissue. The device comprises a bladder having a predetermined size, shape, and material, and a first, and second membrane. A pressurized medium is disposed intermediate the first membrane and the second membrane of the bladder for controlling the environmental conditions surrounding the tissue, and for applying controlled pressure to the tissue. A transfer means is disposed on the bladder to allow the pressurized medium to communicate with the bladder. Controlled pressure can be applied to the tissue to promote healing.

In still a further aspect, the present invention provides a device for treating and promoting the healing of damaged body tissue. The device comprises a bodysuit of a predetermined size, shape, and material for enclosing at least a portion of a body. A transfer means is disposed on the bodysuit to allow at least one of a predetermined medium to communicate with the bodysuit without removing the bodysuit, whereby the tissue can be effectively treated and healed in controlled environmental conditions.

### OBJECTS OF THE INVENTION

It is therefore the primary object of the present invention to provide a device for treating and promoting healing of damaged body tissue.

Another object of the present invention is to provide a device for treating and promoting healing of damaged body tissue by controlling the environmental conditions surrounding the damaged body tissue.

Still a further object of the present invention is to provide a device for treating and promoting healing of damaged body tissue whereby the tissue can be effectively treated with a minimum of pain experienced by the patient.

In addition to the various objects of the invention that have been described above, various other objects and advantages of the invention will become more readily apparent to those persons skilled in the relevant art from the following more detailed description of the invention, particularly, when such description is taken in conjunction with the attached drawing figures and the appended claims.

### BRIEF DESCRIPTION OF THE DRAWINGS

FIG. 1 is a side elevational section view of the present invention;

FIG. 2 is a side elevational section view of an alternative embodiment of the present invention; and

FIG. 3 is a top view of another alternative embodiment of the present invention.

### BRIEF DESCRIPTION OF A PRESENTLY PREFERRED AND VARIOUS ALTERNATIVE EMBODIMENTS OF THE PRESENT INVENTION

Prior to proceeding to a more detailed description of the invention, it should be noted that identical components having identical functions have been designated with identical reference numerals for the sake of clarity.

Now refer more particularly to FIG. 1 of the drawings. Illustrated therein is a device, generally designated 10, for treating and promoting healing of damaged body tissue 12. The device 10 comprises an encapsulating means 14 disposed on the body tissue 12 for controlling the environmental conditions surrounding the tissue. The encapsulating means 14 creates a sterile environment, and further allows for the temperature and humidity to be controlled for the patient's comfort, and to enhance the healing process. Preferably, the encapsulating means 14 is a bandage of a predetermined size, shape, and material. Preferably, the material is transparent, so that the damaged tissue 12 can be viewed directly through the bandage without having to remove the bandage. It is also preferable to have an adhesive 16 disposed on the perimeter of at least one side of the bandage. The adhesive 16 will secure the bandage around the damaged tissue 12, sealing the affected area from exterior environmental conditions.

The device also comprises a transfer means, generally designated 18, disposed on the encapsulating means 14 to allow at least one of a predetermined medium 20 to communicate with the encapsulating means 14 and the tissue 12 without having to remove the encapsulating means 14. Preferably, the transfer means 18 is at least one port of a predetermined size and shape. It is preferred that the transfer means 18 is at least one inlet port 22, and one outlet port 24. Preferably, the medium 20 that communicates with the encapsulating means 14 and the tissue 12 is at least one of a liquid and a gas.

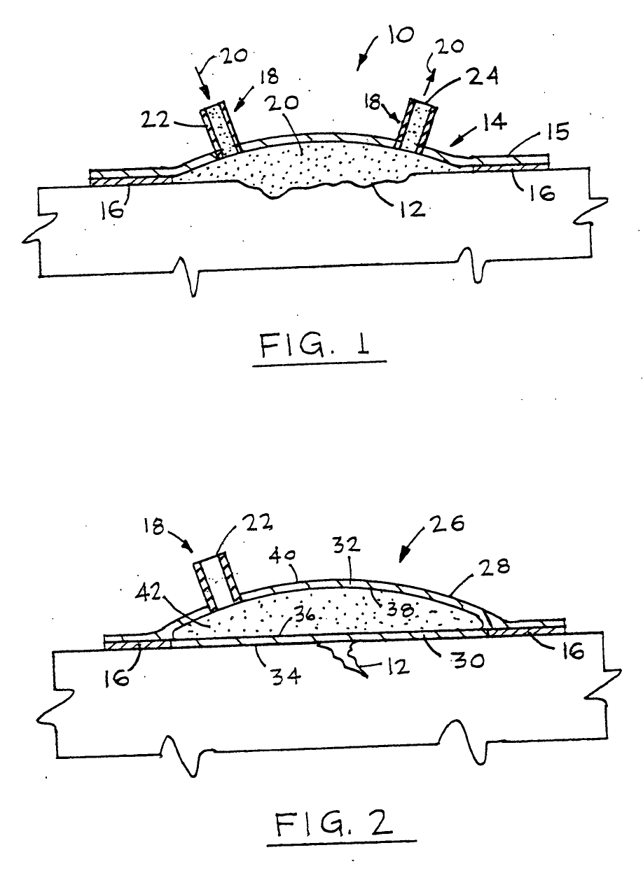

Now refer more particularly to FIG. 2 of the drawings. Illustrated therein is a device, generally designated 26, for treating and healing of damaged body tissue 12. The device 26 comprises a bladder 28 having a predetermined size, shape, and material. Preferably, the bladder 28 has a first membrane 30 and a second membrane 32. The second membrane 32 has a predetermined greater elasticity than such first membrane 30 for enabling such device 26 to act as a tourniquet. The first membrane 30 further has a first surface 34 and a second surface 36, and the second membrane 32 has a third surface 38 and a fourth surface 40. The first surface 34 of the first membrane 30 is disposed on the tissue 12. Preferably, the material of the bladder 28 is transparent so that the damaged tissue 12 is visible through the bladder. It is also preferred that an adhesive 16 is disposed on the perimeter of at least one of the first surface 34 of the first membrane 30 and the fourth surface 40 of the second membrane 32, for securing the bladder 28 to the area surrounding the damaged tissue 12. Preferably, a pressurized medium 42 is disposed intermediate the second surface 36 of the first membrane 30 and the third surface 38 of the second membrane 32 for controlling the environmental conditions surrounding the damaged tissue 12, and for applying controlled pressure to the tissue 12. It is preferred that the pressurized medium 42 is at least one of a gas and a liquid. A transfer means, generally designated 18, is disposed on the bladder 28 to allow the pressurized medium 42 to communicate with the bladder 28, whereby controlled pressure can be applied to the damaged tissue 12 to seal the tissue 12 and promote healing. Preferably, the transfer means 18 is at least one inlet port 22 of a predetermined size and shape.

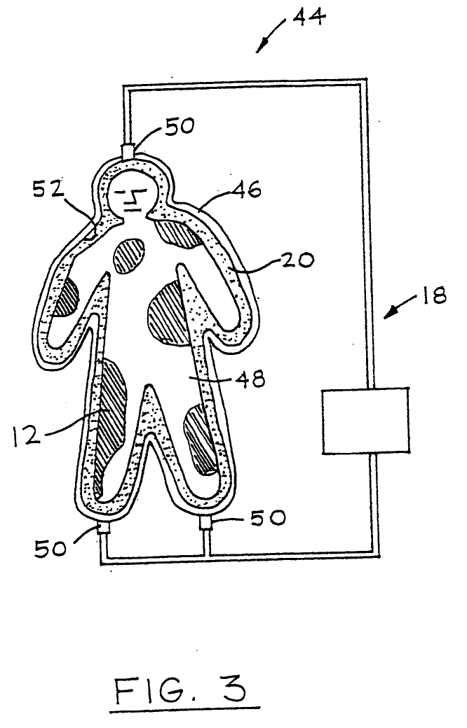

Now refer more particularly to FIG. 3 of the drawings. Illustrated therein is a device, generally designated 44, for treating and promoting healing of damaged body tissue 12. The device 44 comprises a bodysuit 46 of a predetermined size, shape, and material for enclosing at least a portion of a body 48 when larger, and multiple areas of tissue are damaged. Preferably, the material of the bodysuit 46 is transparent to allow the body to be viewed through the bodysuit 46. It is also preferable to have a texture on the inside surface 52 of the bodysuit 46 to permit fluid circulation around the body area that directly contacts the bodysuit 46 when the patient is lying down. A transfer means, generally designated 18, is disposed on the bodysuit 46 to allow at least one of a predetermined medium 20 to communicate with the bodysuit 46 without removing the bodysuit 46, whereby the tissue 12 can be effectively treated and healed in controlled environmental conditions. Preferably, the transfer means 18 is at least one port 50 of a predetermined size and shape, and a circulation pump 54. However, it is preferred that the transfer means 18 includes a plurality of ports 50 to control temperature, humidity, and to circulate liquids, medications, and pain killers around the body 48. It will also be obvious to the reader that a bodysuit 46 that envelops the entire body will have additional ports for feeding and the elimination of bodily waste.

### Operation

Damaged body tissue 12, such as a burn or wound, on a portion of the body 48 is covered with the encapsulating means 14, or bandage. The bandage is sealed around the damaged body tissue 12 by the adhesive disposed on the perimeter of the bandage. Antibiotics and other appropriate medications are injected into the inlet port 22. Inserting a suitable pharmaceutical liquid through the inlet port 22, and extracting the liquid through the outlet port 24 cleanses the affected area. Medication is then reapplied to the clean tissue 12 through the inlet port 22. The bandage remains in place throughout the healing process, maintaining a sterile, humidity and temperature controlled environment. When deeper penetration is required, medications and pain relievers can be applied under pressure to the specific area.

The bladder 28 exerts pressure on a wound, similar to a tourniquet, but in a controlled manner. The pressure in the bladder 28 can be regulated to apply the necessary force required to decrease blood loss.

The bodysuit 46 encapsulates a larger area of damaged body tissue 12 simultaneously. Temperature and humidity can be controlled in the bodysuit 46, while medications and pain relievers can be circulated around the entire area to treat the damaged body tissue 12 and promote healing. In specific circumstances, the encapsulated area can be submerged in water to relieve the pressure on the damaged tissue. The affected area remains sterile and dry. When a full bodysuit 46 is required, the bodysuit 46 can be designed to provide breathing apparatus and elimination of bodily wastes.

Although the invention has been shown in connection with a certain specific embodiment, it will be readily apparent to those skilled in the art that various changes in form and arrangement of parts and method may be made to suit requirements without departing from the spirit and scope of the invention.

1. A device for both treating and healing damaged body tissue, said device comprising:
(a) an encapsulating means, having a predetermined size and a predetermined shape which are at least sufficient to cover such damaged body tissue, disposed adjacent such damaged body tissue for controlling environmental conditions surrounding such damaged body tissue, said encapsulating means including an outer surface and an inner surface;
(b) an adhesive member disposed between and in intimate contact with an outer edge of said inner surface of said encapsulating means and undamaged body tissue surrounding such damaged body tissue to enable adhering said encapsulating means to such undamaged body tissue; and
(c) at least one fluid transfer means sealingly engaged with said encapsulating means and disposed in fluid communication with a space located between said inner surface of said encapsulating means, an inner edge of said adhesive member and such damaged body tissue for allowing communication of at least one predetermined fluid medium to such space disposed between said inner surface of said encapsulating means thereby enabling such damaged body tissue to be effectively treated and healed in controlled environmental conditions.

2. The device, according to claim 1, wherein said encapsulating means is formed from a predetermined material.

3. The device, according to claim 2, wherein said predetermined material is transparent to enable viewing such damaged body tissue during treatment without having to remove said device.

4. The device, according to claim 1, wherein said adhesive member is double sided tape.

5. The device, according to claim 1, wherein said at least one fluid transfer means is at least one port having each of a predetermined size and a predetermined shape.

6. The device, according to claim 5, wherein said device includes at least one inlet port and at least one outlet port so that said at least one predetermined fluid medium can be circulated around such damaged body tissue.

7. The device, according to claim 1, wherein said least one predetermined fluid medium is a liquid medium.

8. A device for each of treating, healing and acting as a tourniquet on damaged body tissue, said device comprising:
(a) a bladder member having each of a predetermined size and a predetermined shape, said bladder member including;
(i) a first member having an outer perimeter and each of a sterile inner surface facing such damaged body tissue and an outer surface, and
(ii) a second member having an outer perimeter secured to said outer perimeter of said first member and each of an outer surface facing atmosphere and an inner surface facing said inner surface of said first member, said second member having a predetermined greater elasticity than said first member for enabling said device to act as said tourniquet;
(b) an adhesive member disposed between and in intimate contact with said inner surface of said first member adjacent said outer perimeter and undamaged body tissue surrounding such damaged body tissue to enable adhering said bladder member to such undamaged body tissue; and
(c) at least one fluid pressure transfer means sealingly engaged with said bladder member and in fluid communication with a space disposed between said first member and said second member for communicating a pressurized fluid into said space and thereby supplying a controlled pressure to said damaged tissue to promote healing thereof.

9. A device for both treating and healing damaged body tissue, according to claim 8, wherein said pressurized fluid is a gas.

10. A device for both treating and healing damaged body tissue, according to claim 8, wherein said bladder member is transparent.

11. A device for both treating and healing damaged body tissue, according to claim 8, wherein said adhesive member is double sided tape.

12. A device for both treating and healing damaged body tissue, according to claim 8, wherein said at least one fluid pressure transfer means is a port.

13. A device for both treating and healing damaged body tissue, according to claim 12, wherein said port has a predetermined size and a predetermined shape.

14. A device for both treating and promoting healing of damaged body tissue, said device comprising;
(a) a bodysuit member having each of a predetermined size and a predetermined shape, said bodysuit having an outer surface facing atmosphere and an inner surface facing a body area of a person to be treated;
(b) a texture formed on said inner surface of said bodysuit member permit circulation of a predetermined fluid around such body area of such person to be treated when such person is lying down;
(c) at least one fluid transfer means sealingly engaged with said bodysuit and disposed in fluid communication with a space located between said inner surface of said bodysuit and such damaged body tissue for allowing communication of at least one predetermined fluid medium to such space disposed between said inner surface of said bodysuit and such damaged body tissue thereby enabling such damaged body tissue to be effectively treated and healed in controlled environmental conditions.

15. A device for both treating and promoting healing of damaged body tissue, according to claim 14, wherein said bodysuit is formed from a transparent material.

16. A device for both treating and promoting healing of damaged body tissue, according to claim 14, wherein said bodysuit includes a plurality of fluid transfer means.

17. A device for both treating and promoting healing of damaged body tissue, according to claim 16, wherein at least one of said plurality of fluid transfer means is an inlet port.

18. A device for both treating and promoting healing of damaged body tissue, according to claim 16, wherein at least one of said plurality of fluid transfer means is an outlet port.

19. A device for both treating and promoting healing of damaged body tissue, according to claim 14, wherein said at least one predetermined fluid medium is a liquid.

20. A device for both treating and promoting healing of damaged body tissue, according to claim 14, wherein said at least one predetermined fluid medium includes medications.

21. A device for both treating and promoting healing of damaged body tissue, according to claim 14, wherein said at least one predetermined fluid medium includes pain killers.

22. A device for both treating and promoting healing of damaged body tissue, according to claim 14, wherein said at least one predetermined fluid medium includes both medications and pain killers.
